(19) United States
(12) Patent Application Publication
(10) Pub. No.: US 2014/0294799 A1
(43) Pub. Date: Oct. 2, 2014

(54) USE OF COMPOSITIONS HAVING A LOW POLYAMINE CONTENT IN THE PREVENTION OR TREATMENT OF ADVERSE EFFECTS LINKED TO CANCER TREATMENT

(71) Applicants: Bernard Cipolla, Saint-Gregoire (FR); Xavier Artignan, Saint-Gregoire (FR)

(72) Inventors: Bernard Cipolla, Saint-Gregoire (FR); Xavier Artignan, Saint-Gregoire (FR)

(73) Assignee: NUTRIALYS MEDICAL NUTRITION SA, St Gregoire (FR)

(21) Appl. No.: 14/241,694

(22) PCT Filed: Aug. 30, 2012
(86) PCT No.: PCT/FR2012/051959

§ 371 Date: Jun. 16, 2014

(30) Foreign Application Priority Data
Aug. 30, 2011 (FR) 11/57646

(51) Int. Cl. A61K 31/131; A23L 1/29; A61K 45/06; A61K 38/51; A61K 38/45

(52) U.S. Cl. CPC A61K 31/131; A61K 38/51; A61K 38/45; A61K 45/06; A23L 1/296
USPC 424/94.5; 514/674; 514/673

(57) ABSTRACT

The composition includes, per gram of composition, less than 600, in particular less than 400 picomoles of biologically active polyamines, for use in the prevention or treatment of radiotherapy-induced pathologies of the skin or mucous membranes in a patient, in particular a human or an animal.

The present invention relates to the use of a composition with a low polyamine content in the prevention or treatment of pathologies following an anti-cancer treatment.

The toxicity of the anti-cancer treatments used within the context of oro-digestive tract cancers leads to significant disturbances in the gastro-intestinal tract of patients. Among the undesirable effects and complications of anti-cancer therapies, mucositis (or inflammation of the mucus) certainly constitutes one of the most debilitating side effects which can lead either to discontinuation or modification of the anti-cancer treatment, which directly results in an increase in patient morbidity or mortality. Oral mucositis is a complication developed by 40% of the patients receiving conventional chemotherapy, and by 75% of those exposed to high-dose chemotherapy before a bone marrow transplant. Approximately 80-90% of the patients treated with radiotherapy for cancer of the head and neck develop oral mucositis. The complications and sequelae of irradiation (mucositis and dermatitis) appear as from the 15th day following the start of the radiotherapy and persist throughout the duration of the treatment.

Although the frequency and intensity of mucositis vary depending on the nature and duration of the treatment (chemotherapy and/or radiotherapy), genetic or tissue factors, their development always follows a sequence comprising five phases: initiation, the genesis of a signal, amplification of the signal, ulceration and resolution (Sonis et al., 2004, J Support Oncol, 2(1), 21-36; Bowen et al., 2008, Journal of Oncology, 2008, 1-7). The initiation results from direct damage to the DNA and other cell components of the epithelium and of the sub-mucosa caused by the anti-cancer treatments. This deterioration leads to a cascade of activation of transcription factors, in particular of NFκB which regulates the expression of numerous genes, including those involved in the synthesis of pro-inflammatory cytokines (TNF-α, IT-1β, IT-6),

of adhesion molecules and synthesis pathways involved in the apoptosis of the epithelial cells. In turn, these cytokines activate NFκB as well as other signalling pathways, thus creating a signal amplification loop leading to ulceration, i.e. the destruction of the epithelium and part of the sub-mucosa. The ulcerated surface can be colonized by toxin-producing bacteria. This stage of ulceration is at the origin of the symptoms of mucositis: pain, inflammation and loss of the protection function of the mucous membrane vis-à-vis the outside environment. The resolution of the mucositis consists of the migration, growth and differentiation of precursors of epithelial cells over the ulcerated area. This resolution phase generally occurs spontaneously in the days following the discontinuation of the anti-cancer treatment.

The severity of mucositis is classified from grade 1 (asymptomatic or painless erythema) to grade 5 (death). Grade 1 corresponds to the onset of erythema of the mucous membrane. Grade 2 corresponds to the development of ulcerations or isolated pseudomembranes. As from grade 3, the ulcerations or pseudomembranes join up and can lead to slight bleeding. Necrosis of the tissues is visible in grade 4 resulting in bleeding. Grades 3 and 4 must be treated rapidly insofar as the patient suffering from bleeding ulcerations accompanied by severe pains can generally no longer be fed normally. Feeding by enteral or parenteral route is necessary in order to avoid any risk of malnutrition.

In the treatment of cancers affecting the oro-digestive tracts, the tissues affected by mucositis are those of the oral cavity (tongue, cheeks, gums, palate), the pharynx, oesophagus, stomach and all of the digestive tract as far as the rectum (small intestine, colon). However, the intrinsic properties of these tissues confer upon them a variable ability to respond to anti-cancer treatment in terms of times of onset, duration and intensity of mucositis. In fact, intestinal mucositis occurs earlier than oral mucositis in a patient treated with 5-fluorouracil (5-FU). This is probably a consequence of the single-layer structure of the intestinal epithelium whereas the oral mucous membrane is constituted by a stratified epithelium. Other factors such as the composition of the biological fluids

lining the different epithelia or the three-dimensional organization of the latter can also influence the onset of mucositis.

The treatment of mucositis resulting from an anti-cancer treatment remains empirical and is based mainly on mouthwashes, the use of anti-bacterial agents or non-steroidal anti-inflammatories (paracetamol, salicylic acid), or also on cryotherapy (ice in the oral cavity) following administration of a bolus of anti-cancer agent or a radiation session (Keefe et al., 2007, American Cancer Society, 820-831). In response to the relative efficacy of such treatments, there is a real need for novel drugs that can be used in the treatment of mucositis induced by anti-cancer treatment.

At present, Kepivance® (palifermin) constitutes the only medicinal product approved by the health authorities (FDA, ANSM) indicated for reducing the incidence, duration and severity of oral mucositis only in patients suffering from malignant blood disease. Kepivance® is a human keratinocyte growth factor (KGF) which stimulates the proliferation and differentiation of the epithelial cells. Its very restricted indication limits the number of patients who can benefit from this treatment.

The document U.S. Pat. No. 7,045,550 discloses the use of compositions containing natural polyamines (agmatine, spermine, spermidine, cadaverine, putrescine) making it possible to reduce certain side effects developed under anti-cancer treatment. The topical application of these compositions to the skin of irradiated animals shows that agmatine makes it possible to reduce dermatitis whereas spermine is effective in the treatment of alopecia.

Recent studies relating to the physiopathology of mucositis have allowed the development of new treatments targeting in particular one or other of the stages of the development of mucositis.

The document WO 2011/064297 discloses oral compositions comprising a mixture of glycine, proline optionally combined with lysine, leucine and a synthetic or natural polymer capable of forming a film. These compositions, when they are applied locally to patients having undergone radio- or

chemotherapy, improve the clinical appearance of lesions and also reduce the inherent pain of oral mucositis.

The intracellular signalling pathways also constitute an important pharmacological target. Thus, the use of direct inhibitors (Resveratrol, U.S. Pat. No. 6,841,578), or indirect inhibitors (histone deacetylase inhibitor, document US 2006/0275370) of the activation of the transcription factor NFκB prevents the setting up of the signal amplification loop induced by TNF-α, thus preserving the epithelial cells from apoptosis. The patent EP 1 704 860 discloses pharmaceutical compositions containing a benzamidine derivative capable of inhibiting the synthesis of the pro-inflammatory cytokines IT-1β, IT-6 and TNF-α as well as the expression of enzymes involved in inflammation (inducible cyclooxygenase-2 and NO synthase) which also participate in the signal amplification loop and in the adverse effects of anti-cancer treatments.

Other teams have been interested in the ulceration stage, in particular in the presence of bacteria. U.S. Pat. No. 7,727,974 discloses compositions and their use in the prevention or treatment of mucositis of the digestive tract induced by radio- or chemotherapy. These compositions include an antagonist of toll-like receptor 4 (TLR4) involved in signal transduction not only of the lipopolysaccharide (LPS) originating from the bacteria walls, but also in that of the heat shock proteins (HSP60, 70 and 90).

Another strategy consists of specifically targeting the epithelial cell. U.S. Pat. No. 6,764,683 deals with the use of peptides derived from TGF-α, involved in the proliferation and migration of the precursors of the epithelial cells of the mucous membrane lining the gastro-intestinal tract. The document WO 2006/122162 discloses compositions comprising peptides derived from the protein AMP-18, a protein synthesized in the stomach and involved in the homeostasis of the epithelial cells lining it. In the document US 2008/0026058, the methods for treating mucositis by oral or local route which are described, are based on the use of pigments or natural tannins, in particular the anthocyanisodes or the flavonols, which play a role in the healing of the epithelium.

The document WO 2008/110585 shows that the treatment of radio-induced mucositis with a composition comprising 5-[2-pyrazinyl]-4-methyl-1,2,3-thione, or one of its derivatives, optionally combined with N6-isopentenyl adenosine, or with one of its derivatives, leads to complete protection of irradiated animals vis-à-vis the gastrointestinal syndrome of which mucositis forms part.

The use of an anti-tumour agent in combination with a polysaccharide (document WO 2011/016111) or a fatty acid (document WO 2011/031210) makes it possible not only to reduce the grade of mucositis but also to reduce the toxicity of the anti-tumour agent.

Although certain agents have been mentioned as being able to speed up recovery and modify the progression of mucositis, at present there are no treatments with established efficacy making it possible to prevent or treat mucositis satisfactorily.

The inventors of the present invention have shown that a polyamines-depleted diet, replacing a normal diet, made it possible not only to reduce the volume of the tumour (EP 0 703 731 B1) but also to reduce pain perception (EP 1 648 431 B1).

The purpose of the present invention is to propose a method for preventing or treating mucositis and/or of dermatitis induced by radiotherapy and/or chemotherapy.

Another objective of the invention is to provide such a method of prevention and treatment having no or very few side effects.

Another objective of the invention is to provide such a method of prevention and treatment, in order to limit the pauses, the dose reductions or the risk of discontinuing radiotherapy when used alone or used in combination with chemotherapy, and therefore to increase the patient's chances of survival.

The present invention relates to a composition comprising, per gram of composition, less than 600, in particular less than 400 picomoles of biologically active polyamines, for use in the prevention or treatment of

radiotherapy-induced pathologies of the skin or mucous membranes in a patient, in particular a human or an animal.

The present invention relates to a composition comprising a mixture of at least two natural polyamines chosen from spermine, spermidine, putrescine or cadaverine and comprising, per gram of composition, less than 600, in particular less than 400 picomoles of biologically active polyamines, for use in preventing or treating radiotherapy-induced mucositis, salivation problems (dry mouth/pathology of the mucous membranes and salivary glands) and pain on swallowing, that can occur in the case of the highest grades of radiotherapy-induced mucositis, and radiotherapy-induced dermatitis, in a patient, in particular a human or an animal.

Surprisingly, the authors have demonstrated that a low-polyamine diet made it possible to prevent or delay the onset of mucositis or to treat mucositis following an anti-cancer treatment.

The use of a low-polyamine composition thus allows a triple action, namely synergy with the anti-tumour treatment, treatment of the pain and the prevention and/or treatment of the side effects induced by radiotherapy.

The compositions of the present invention can be used as a food substitute (nutraceutical composition), or as a food supplement, intended for a patient or an animal.

By “food substitute” is meant a composition consumed instead of any other form of food. A food substitute is intended to replace a normal food while ensuring the daily intake of nutrients. This type of diet is described as a strict diet.

It should be noted that in the case of a strict diet, all the biologically active polyamines absorbed daily originate from the nutraceutical composition. This makes it possible to advantageously monitor the patient's or animal's daily polyamine intakes.

By “food supplement” is meant a composition consumed in addition to normal food. A food supplement is added to the normal diet. The normal diet can be modified in order to take into account the food supplement intakes so as not to disturb the nutritional balance. By “modification of the normal diet” is meant a reduction in the quantities of one or more or all foods, or a modification of the nature of the foods absorbed.

By “polyamines”, is meant the organic compounds having two or more amine functions and having a biological activity.

By “biologically active polyamines”, is meant the polyamines which have an effect on:

the stabilization, condensation and conformation of the DNA (Thomas & Thomas (2001) “Polyamines in cell growth and cell death: molecular mechanism and therapeutic applications.” CMLS, Cell Mol Life Sci 58: 244-258),

RNA transcription,

cell growth and proliferation by acting directly on the cell cycle of the cells (Thomas & Thomas 2001),

regulation of the immune response (Soulet D & Rivest S (2003) “Polyamines play a critical role in the control of the innate immune response in the mouse central nervous system.” The Journal of Cell Biology vol. 162; No. 2 July 21: 257-268),

modulation of the operation of the N-methyl-D-aspartate (NMDA) receptors and are involved in the neurodegeneration process (Soulet & Rivest 2003).

The biologically active polyamines are the polyamines (putrescine, cadaverine, spermine and spermidine) present in the food composition mixture without being chelated, protected, or masked.

The biologically active polyamines can be identified in particular by at least one of the following methods:

1) In Culture:

A biologically “active” polyamine or one of its derivatives must be able to participate in the physiological cell metabolism of the polyamines, or even be capable of interfering with the latter or even dysregulating it.
a. A biologically “active” polyamine must therefore be able to be associated with or even be recognized by the transport system(s) for internalizing it into a living cell. The addition of a radio-labelled biologically “active” polyamine to the culture medium makes it possible to verify its internalization.
b. A biologically “active” polyamine must be able to suppress the inhibition of cell proliferation caused by the inhibition of the endogenous anabolism of the polyamines (e.g.: by α-DFMO).
c. A biologically “active” polyamine, including synthetic polyamine, by dysregulating the natural metabolism of the polyamines must be able to modulate the level of cell proliferation.

2) In Vivo:

In an animal bearing a transplanted tumour, the exogenous supply (gastro-intestinal tract) of “active” polyamines must suppress the beneficial anti-cancer effects caused by the deficiency in “active” polyamines induced by the reduction of the endogenous and exogenous sources of “active” polyamines, this exogenous supply being coupled or not coupled with anti-cancer medicinal products.

By “prevention or treatment”, is meant the ability of the composition to prevent, delay or moderate the onset of pathologies of the skin or of the mucous membranes induced by the radiotherapy and/or to reduce the intensity of these pathologies, when they are already present in the patient, from a high grade to a lower grade.

By “skin or mucous membranes”, is meant the tissues constituted by a dermis and an epidermis as well as the tissues constituted by an epithelium that is, to a greater or lesser extent, stratified.

By “pathologies of the skin”, is meant inflammation of the skin due to exposure to ionizing radiation. This inflammatory reaction is manifested in particular by erythema, blistering, skin lesions, or ulceration.

By “pathologies of the mucous membranes”, is meant an inflammation of the mucous membranes associated with a burning or tingling sensation. This inflammation is characterized by atrophy of the squamous epithelium, vascular damage, an infiltration of inflammatory cells and ulceration.

The pathologies to which the present invention relates are in particular mucositis, salivation problems (dry mouth/pathology of the mucous membranes and salivary glands) and pain on swallowing, which can occur in the case of the highest grades of mucositis, and dermatitis.

By “radiotherapy”, is meant any method of radiation leading to the irradiation of the patient. The radiation can be obtained by a linear high-energy particle accelerator or by one or more electron beams focussed and directed locally onto the zone to be treated. The radioactive source can also be in liquid, injectable form and be fixed to the target cells or in solid form, placed in the patient's body for a given period of time. Radiotherapy comprises three main techniques:

1—external radiotherapy: this is the best-known and the most-used; the radiation source is located outside the patient. Linear high-energy X-ray particle accelerators and electron beams are preferably used. There are three main techniques of external radiotherapy: conventional radiotherapy, conformational radiotherapy and tomotherapy or helical radiotherapy.

2—curietherapy: the radioactive source is placed, for a limited period of time (usually a few hours) or permanently, inside the patient's body, namely in the tumour or in a cavity in contact with it. At present there are three main techniques, themselves being subdivided into sub-techniques depending on the radiation dosage rate (low rate and high rate) and the type of application (manual application or remote afterloading). These are interstitial curietherapy, endocavity curietherapy and endoluminal curietherapy.

3—vectorial metabolic radiotherapy: the radioactive source is liquid, injectable, unsealed, and is fixed to the target cells.

The present invention also relates to a composition comprising, per gram of composition, less than 600, in particular less than 400 picomoles of biologically active polyamines for use in the prevention or treatment of radiotherapy-induced pathologies of the skin or mucous membranes, in a patient, in particular a human or an animal, the total quantity of biologically active polyamines ingested by the patient daily not exceeding 0.40 nanomoles per kcal of composition ingested, in particular 0.30 nanomoles per kcal of composition ingested, in particular 0.25 nanomoles per kcal of composition ingested, in particular 0.20 nanomoles per kcal of composition ingested.

A normal diet providing a daily intake of 2000 kcal (kilocalories), for an adult weighing 70 kg, can contain, depending on the foods consumed, from 200,000 to 700,000 nmol of biologically active polyamines per day, i.e. from 100 to 350 nmol of biologically active polyamines per kilocalorie per day.

A standard food provides an intake of 250 nmol of biologically active polyamines per kilocalorie per day (Bardocz et al., 1995, Br J Nutr, 73(6):819-828).

The intention of the present invention is not to completely eliminate the intake of biologically active polyamines from the patient's diet but to reduce the intake thereof, i.e. to considerably reduce the concentrations of exogenous biologically active polyamines and optionally to inhibit the synthesis of the endogenous biologically active polyamines. Thus, the compositions used according to the present invention still comprise biologically active polyamines but at low levels in comparison with those originating from a standard food.

The compositions used according to the present invention comprise a minimum of 20 picomoles of biologically active polyamines per gram of composition. In other words terms, the compositions used according to the present invention comprise levels of biologically active polyamines which vary from 20 picomoles per gram of composition to less than 600 picomoles per gram of composition.

The biologically active polyamines are present in the compositions of the invention at levels 100 times lower, in particular 500 times lower, in particular 1000 times lower than the quantity of biologically active polyamines present in a normal diet. This means that the biologically active polyamines are present in the compositions used according to the present invention at a level of from 1 to 3.5 nmol per kilocalorie per day, in particular from 0.2 to 0.7 nmol per kilocalorie per day, in particular from 0.10 to 0.35 nmol per kilocalorie per day. In particular, the biologically active polyamines are present in the compositions used according to the present invention at a level of from 0.1 to 0.4 nmol per kilocalorie per day, in particular from 0.1 to 0.2 nmol per kilocalorie per day, in particular from 0.2 to 0.3 nmol per kilocalorie per day, in particular from 0.3 to 0.4 nmol per kilocalorie per day.

In order to be able to convert quantities expressed in nanomoles of polyamines per kilocalorie to grams of polyamines per kilocalorie, it is necessary to consider an average molecular weight for all of the biologically active polyamines. This average molecular weight of the biologically active polyamines is an approximation necessary for carrying out this calculation; it is estimated at 145.24 g/mol.

This means that the biologically active polyamines are present in the compositions used according to the present invention at a level of from 14.5 to 508.3 ng per kilocalorie per day. In particular, the biologically active polyamines are present in the compositions used according to the present invention at a level of from 14.5 to 58 ng per kilocalorie per day, in particular from 14.5 to 29 ng per kilocalorie per day, in particular from 29 to 43.5 ng per kilocalorie per day, in particular from 43.5 to 58 ng per kilocalorie per day.

The pathologies to which the present invention relates are pathologies of the skin or of the mucous membranes belonging to the following group: mucositis, dermatitis.

By “mucositis”, is meant the inflammation of the mucous membrane of the whole of the gastro-intestinal tract extending from the mouth to the rectum. When the mucositis is located in the upper aerodigestive tracts (oral cavity, nasal passage, pharynx), the terms mucositis or stomatitis denote the same pathology and are interchangeable.

By “dermatitis”, is meant any inflammatory reaction of the skin due to exposure to ionizing radiation. Skin with dermatitis is warmer than areas of healthy skin. The dermatitis can occur in the form of redness (erythema) in the case of the least serious forms or in the form of ulcerous lesions in the case of the most serious forms.

According to a particular embodiment, the polyamines of the composition used according to the present invention are a mixture of at least two natural polyamines chosen from spermine, spermidine, putrescine or cadaverine. This means that the compositions used according to the present invention comprise spermine and spermidine, or spermine and putrescine, or spermine and cadaverine, or spermidine and putrescine, or spermidine and cadaverine, or putrescine and cadaverine, or spermine and spermidine and cadaverine, or spermine and spermidine and putrescine, or spermidine and putrescine and cadaverine, or spermine and spermidine and putrescine and cadaverine.

According to a more particular embodiment, the composition used according to the present invention contains, per gram of composition, less than 300, in particular less than 200 picomoles of putrescine, less than 50, in particular less than 20 picomoles of spermine, less than 150, in particular less than 100 picomoles of spermidine, less than 100, in particular less than 80 picomoles of cadaverine.

According to another embodiment, the composition used according to the present invention contains, per gram of composition, less than 400 picomoles of polyamines.

In particular, the composition used according to the present invention contains, per gram of composition, less than 200 picomoles of putrescine, less than 20 picomoles of spermine, less than 100 picomoles of spermidine, less than 80 picomoles of cadaverine.

The composition can be used according to the present invention in a patient, said patient being able to be a be human or an animal.

The grade of mucositis or dermatitis can require a particular packaging of the composition used according to the present invention. In the case of dermatitis, it is possible to envisage administration by topical route (cream, gel or ointment).

According to a particular embodiment, the composition used according to the present invention is packaged in a form of a dose suitable for administration to the patient, in particular by systemic route.

In the case of grade 3 or 4 oral mucositis, the composition must be administered by systemic route, in particular by enteral or parenteral route.

The dose of polyamines received by the patient can be adapted depending on the anti-cancer treatment considered, the seriousness of the lesions caused by said treatment, the duration of said envisaged treatment as well as the tolerance of the patient receiving said treatment.

The composition can be used according to the present invention in a patient for whom the treatment is carried out by administering a unit dose varying from 10 μg to 20 μg of polyamines.

Radiotherapy does not constitute the only method for treating cancer. Surgery and chemotherapy are also commonly used. The therapeutic strategy is implemented by the hospital practitioner who can choose to combine several of these methods of treatment. Over the last few decades, chemotherapy and radiotherapy combined with surgery have effectively contributed to a significant reduction in deaths from cancer. However, the potential usefulness of the medicinal products used in chemotherapy as well as radiotherapy in the treatment of cancer has not been fully exploited because of undesirable effects associated with non-specific cytotoxicity of these agents or treatments.

According to a particular embodiment, the compositions of the present invention are used for preventing or treating in a patient, pathologies of the skin or of the mucous membranes induced by radiotherapy combined with either a chemotherapeutic agent, or with surgery, or with a chemotherapeutic agent and surgery.

According to a particular embodiment, the compositions of the present invention are used for preventing or treating pathologies of the skin or of the mucous membranes induced by radiotherapy associated with a chemotherapeutic agent, in a patient.

By “chemotherapeutic agent”, is meant the compounds known to a person skilled in the art for treating cancer. A non-exhaustive list of these compounds is given by way of indication but must not serve to restrict the general concept of the invention. These compounds can be chosen from the alkylating agents (busulphan, carboplatin, chlorambucil, oxaliplatin, cisplatin, cyclophosphamide, ifosfamide, melphalan, mechlorethamine, oxaliplatin, uramustine, temozolomide), the antimetabolites (azathioprine, capecitabine, cytarabine, floxuridine, fludarabine, fluorouracil, gemcitabine, methotrexate, pemetrexed), the plant alkaloids (vinblastine, vincristine, vinorelbine), the topoisomerase inhibitors (irinotecan, topotecan, etoposide), the anti-tumour antibiotics (bleomycin, daunorubicin, doxorubicin, epirubicin, hydroxyurea, idarubicin, mitomycin C, mitoxantrone), the anti-angiogenic agents (cetuximab) and the poisons of the mitotic spindle (docetaxel, paclitaxel, vinblastine, vincristine, vinorelbine).

Concomitant or alternating chemoradiotherapy is therefore the combination of radiotherapy with chemotherapy. It has been, and is still, the subject of intensive clinical research into the treatment of locally advanced tumours (Reboul. et al., Bulletin Cancer, 1999, 86(1), 77-83). This approach has the double objective of acting immediately on occult metastatic dissemination and improving local control by means of the chemotherapy-radiotherapy interactions demonstrated by numerous experimental studies. It is based on the administration, in the case of during radiotherapy, of only one or several drugs active in the tumour considered and having radio-sensitizing properties (cisplatin-5FU in tumours of the ear, nose and throat, and the oesophagus, cisplatin-etoposide in bronchial tumours, 5FU-folinic acid in rectal tumours, 5FU-mitomycin in tumours of the anal canal etc.). The radiotherapy is administered in varying ways, spread over time or fractionated in standard manner, or bifractionated, accelerated or not. This approach generally proves superior to radiotherapy alone in terms of local control, of relapse-free survival and overall survival, whether postoperatively, preoperatively or exclusively in inoperable tumours. In the numerous tumours where concomitant chemoradiotherapy has proved superior to radiotherapy alone or to surgery alone, certain improved protocols have made it possible to reduce toxicity, such as the conformal radiotherapy technique making it possible to better protect healthy tissues.

According to another particular embodiment, the compositions of the present invention are used for preventing or treating in a patient, pathologies of the skin or of the mucous membranes induced by radiotherapy combined with surgery.

By “surgery”, is meant the standard techniques of intervention by a specialist practitioner, on the tissues of the patient. The purpose of this method is to allow a complete exeresis of the tumour by performing a resection from health tissue while aiming to preserve the function of the organ or of the tissue affected. The surgical stage is performed on a physically accessible tumour and generally occurs before the start of the radiotherapy.

There is also another technique called radiosurgery. This is a non-invasive technique making it possible to treat benign tumours or cancers without resorting to surgery. It consists of irradiating an area in a very precise manner. Radiosurgery differs from conventional radiotherapy in that the aim is not a differential effect (conventional radiotherapy uses the fractionation of the dose to allow the repair of healthy tissues) but localized necrosis of the target tissue. Radiosurgery does not come within the scope of the invention as it does not induce mucositis.

For certain resistant or invasive cancers, combination of the three methods of treatment can prove necessary.

According to yet another particular embodiment, the compositions of the present invention are used for preventing or treating, in a patient, pathologies of the skin or of the mucous membranes induced by radiotherapy combined with a chemotherapeutic agent and with surgery.

When the compositions used according to the invention constitute the patients' only or main food source, these compositions can be enriched with lipids, proteins, carbohydrates, vitamins, minerals and electrolytes in quantities allowing the patient not to suffer from malnutrition or deficiencies.

The composition used according to the present invention contains, as a percentage of dry weight with respect to the total dry weight: 10% to 35% lipids, 8% to 30% proteins, 35% to 80% carbohydrates, up to 10% of a mixture constituted by vitamins, minerals and electrolytes.

By “food source” is meant all forms of diet, i.e. all of the foods that can make up the diet of a patient or animal, a diet comprising meal replacements, or any other source of nutrition making it possible to keep the patient or the animal alive, while avoiding foods containing polyamines.

The organism's polyamines originate from three main sources: cell proliferation (physiological and tumour), food and the intestinal bacteria. For maximum control of the supply of polyamines to the organism, it can prove necessary to limit not only the exogenous supply via a perfectly controlled food but also to reduce the intracellular supply of polyamines in particular by inhibiting the endogenous synthesis of cellular origin of the polyamines or by blocking the transport of the polyamines which takes place between the cell and the extracellular medium.

The endogenous synthesis of the polyamines is based on the use of specific inhibitors. By “specific inhibitor”, is meant a molecule capable of blocking, completely or partially, directly or indirectly, reversibly or irreversibly, the active site of at least one of the enzymes involved in the synthesis of the polyamines (ornithine decarboxylase (ODC), spermidine-spermine N1-acetyltransferase or spermine oxydase). The role of the polyamine biosynthesis inhibitor is to stop of significantly reduce the endogenous production of polyamines in the organism treated with the product according to the present invention. The combined use a polyamine synthesis inhibitor and a food intake with a low polyamines content makes it possible to reduce the quantity of bio-available polyamines in the organism.

According to another embodiment, the present invention relates to the use of a composition, for use per os, for preparing a medicinal product, which medicinal product contains a quantity of at least one polyamine synthesis inhibitor corresponding to a daily dose, depending on the weight of the patient, of polyamine biosynthesis inhibitor(s) of from approximately 5 to 20 mg/kg/day, in particular 7 to 14 mg/kg/day, and particularly approximately 9 mg/kg/day. These doses are given in the case of a human whose the weight is estimated at 70 kg.

If the patient is an animal, the daily dose of polyamine biosynthesis inhibitor(s) corresponds to approximately 2 to 10 g/kg/day, in particular 3 to 5 g/kg/day (Quenemer et al., 1995, Ann Gastroenterol Hepatol (Paris), 31(3), 181-188; discussion 188-189/Leveque et al., 2000, Anticancer Res, 20(1A), 97-101).

The composition used according to the present invention is enriched with at least one intracellular polyamine synthesis inhibitor, in particular an ornithine decarboxylase, spermidine-spermine N1-acetyltransferase or spermine oxydase inhibitor, in particular at a level of at the most 15% by weight with respect to the total dry weight of the composition.

According to a more particular embodiment, the intracellular polyamine synthesis inhibitor of the composition used according to the present invention is an ornithine decarboxylase, spermidine-spermine N1-acetyltransferase or spermine oxydase inhibitor.

Among the ODC inhibitors, alpha-difluoromethylornithine (α-DFMO) constitutes a useable compound, well known to a person skilled in the art (Fabian et al., 2002, Clin Cancer Res, 8(10), 3105-3117/Levin et al., 2003, Clin Cancer Res, 9(3), 981-990/Meyskens et al., 2008, Cancer Prey Res, 1(1), 32-38). This example must in no event restrict the choice of an endogenous polyamine synthesis inhibitor to this single compound.

Other compounds capable of inhibiting ornithine decarboxylase, spermidine-spermine N1-acetyltransferase or spermine oxydase can be used. The quantities of inhibitors are adapted by a person skilled in the art based on the database of biological activity of these compounds and on their general knowledge.

According to another embodiment, the composition used according to the present invention is enriched with at least one polyamine transport inhibitor, at a level of at the most 15% by weight with respect to the total dry weight of the composition.

The transport of the polyamines between the cell and the extracellular medium also allows fine regulation of the intracellular polyamine content. (Igarashi et al., 2010, Plant Physiology and Biochemistry, 48, 506-512).

The development of polyamine transport inhibitors is the subject of numerous research programmes. Different classes of molecules have been developed, in particular spermine analogues (Burns M. R., 2009, J Med Chem, 52, 1983-1993) or polyamine dimers (US 2005/0267220 A1), optionally linked to an anthracene core (WO 2010/148390). The combined use of a polyamine transport inhibitor and a low-polyamine food supply makes it possible to reduce the quantity of bio-available polyamines in the organism.

In order to still further reduce the endogenous synthesis of polyamines, it is possible to envisage resorting to antibiotics in order to limit the supply of polyamines by the bacteria of the intestinal flora.

According to a particular embodiment, the composition used according to the present invention can contain at least one antibiotic.

The use of antibiotics can lead to a reduction in the supply of certain vitamins in particular those supplied by the intestinal flora of the patient. In this case, it can prove necessary to supplement the composition with these vitamins so as not to cause vitamin deficiencies in the patient in the event of prolonged administration of the composition.

By “deficiencies”, is meant a lack of nutriments that can alter the physical or mental condition of a patient or an animal.

According to a particular embodiment, the composition used according to the present invention can be enriched with vitamins.

According to a particular embodiment, the composition used according to the present invention contains at least one antibiotic and/or is enriched with vitamins

Preferentially, when they are present, the carbohydrates of the composition used according to the present invention belong to the group comprising the glucose polymers, maltodextrins, saccharose, modified starches, glucose monohydrate, dehydrated glucose syrup, glycerol monostearate and mixtures thereof.

Preferentially, when they are present, the proteins of the composition used according to the present invention belong to the group comprising soluble milk proteins, soya proteins, serum peptides, powdered egg white, potassium caseinate, non-phosphorylated peptides, casein peptides, mixed caseinate, soya isolate and mixtures thereof.

Preferentially, when they are present, the lipids of the composition used according to the present invention belong to the group comprising butter oil, groundnut oil, medium chain triglycerides, grape seed oil, soya oil, evening primrose oil and mixtures thereof.

According to a particular embodiment, when they are present the lipids of the composition used according to the present invention are constituted by a mixture of at least one oil of animal origin, at least one oil of vegetable origin and glycerol stearate.

In order to control the supply of polyamines from an exogenous source, the compositions used according to the present invention must be able to constitute all or part of the food of the patient undergoing anti-cancer treatment. To this end they must supply an energy intake capable of satisfying the patient's nutritional requirements. The patient can be a human being or an animal.

When the patient is a human being, the compositions used according to the present invention constitute the human being's daily food ration and comprise:
from 75 g to 500 g of carbohydrates,
from 20 g to 185 g of lipids,
from 20 g to 225 g of proteins,
vitamins, minerals and electrolytes in quantities sufficient to meet the daily nutritional requirements of a human being,
and optionally an intracellular polyamine synthesis inhibitor at a level of less than 50 g and preferentially at a level of 0.3 to 10 g per day.

According to a particular embodiment, the compositions used according to the present invention are a fraction of a human being's daily food ration and comprise:
from 75/X g to 500/X g of carbohydrates,
from 20/X g to 185/X g of lipids,
from 20/X g to 225/X g of proteins,
vitamins, minerals and electrolytes in quantities sufficient to meet the daily nutritional requirements of a human being,
and optionally an intracellular polyamine synthesis inhibitor at a level of less than 50/X g and preferentially at a level of 0.3/X to 10/X g per day,
   and X being an integer comprised between 2 and 8 and corresponding to the number of rations that must be ingested by the patient in order to satisfy their daily nutritional requirements.

When the patient is an animal, the daily food ration is adapted depending on the type and weight of the animal, whether it is a pet or farmed animal. The distribution into carbohydrates, lipids and proteins as well as the requirements for vitamins, minerals and electrolytes of the daily food ration of an animal are well known to a person skilled in the art. With regard to the intracellular polyamine synthesis inhibitor, the dose is adapted depending on the weight of the animal, optionally based on the data obtained from humans.

The compositions used according to the present invention can constitute the daily food ration or a fraction of the daily food ration of an animal and must satisfy the daily nutritional requirements of an animal.

By way of example, when the patient is a mouse, the compositions used according to the present invention constitute the daily food ration of a mouse and comprise:
from 0.6 g to 1.8 g of carbohydrates,
from 0.04 g to 1.2 g of lipids,
from 0.01 g to 0.6 g of proteins,
vitamins, minerals and electrolytes in quantities sufficient to meet the daily nutritional requirements of an animal,
and optionally an intracellular polyamine synthesis inhibitor at a level of less than 300 mg and preferentially at a level of 40 to 200 mg per day.

Just as for a human being, the compositions used according to the present invention can be a fraction of a daily food ration of a mouse and comprise:
from 0.6/X g to 1.8/X g of carbohydrates,
from 0.04/X g to 1.2/X g of lipids,
from 0.01/X g to 0.6/X g of proteins,
vitamins, minerals and electrolytes in quantities sufficient to partially meet the daily nutritional requirements of an animal,
and optionally an intracellular polyamine synthesis inhibitor at a level of less than 300/X mg and preferentially at a level of 40/X to 200/X mg per day,
   and X being an integer comprised between 2 and 8 and corresponding to the number of rations that must be ingested by the patient in order to satisfy its daily nutritional requirements.

The proportions of the constituents of the compositions of the present invention indicated within the context of a mouse's food are given as an indication and can serve as a basis to a person skilled in the art who can adapt them, using their general knowledge, to other animals.

According to the grade of mucositis, the patient can experience difficulties in the absorption of foods, in particular difficulties with chewing and swallowing, or cannot be fed by mouth because of the pain caused by the mucositis. The compositions used according to the present invention can be presented in semi-liquid or liquid form, thus allowing feeding by oral route, but also by enteral or parenteral route.

According to a particular embodiment, the compositions used according to the present invention are presented in dried form to be dissolved extemporaneously in a neutral vehicle.

According to a more particular embodiment, the compositions used according to the present invention include a neutral vehicle making them ready for use.

The sequence of administration of the compositions used according to the present invention can be adapted according to whether the cancer patient is already undergoing treatment or is on the point of receiving an anti-cancer treatment.

The compositions used according to the present invention can be administered to the patient:
before the radiotherapeutic treatment, or
before and during the radiotherapeutic treatment, or
before and after the radiotherapeutic treatment, or
before, during and after the radiotherapeutic treatment, or
during the radiotherapeutic treatment, or
during and after the radiotherapeutic treatment, or
after the radiotherapeutic treatment.

The compositions used according to the present invention can be administered to the patient before and/or during and/or after the radiotherapeutic treatment.

According to a particular embodiment, the compositions used according to the present invention can be administered to the patient before the radiotherapeutic treatment.

According to a more particular embodiment, the compositions used according to the present invention can be administered to the patient before and during the radiotherapeutic treatment.

According to another particular embodiment, the compositions used according to the present invention can be administered to the patient before and after the radiotherapeutic treatment.

According to another more particular embodiment, the compositions used according to the present invention can be administered to the patient before, during and after the radiotherapeutic treatment.

According to another particular embodiment, the compositions used according to the present invention can be administered to the patient during the radiotherapeutic treatment.

According to another more particular embodiment, the compositions used according to the present invention can be administered to the patient during and after the radiotherapeutic treatment.

According to another even more particular embodiment, the compositions used according to the present invention can be administered to the patient after the radiotherapeutic treatment.

In order to increase the chances of remission of the cancer in a patient, the compositions used according to the present invention can be administered to the patient having undergone radiotherapy in combination with a second cancer treatment procedure. The second cancer treatment procedure is chosen from chemotherapy and/or surgery.

According to a particular embodiment, the compositions used according to the present invention can be administered to the patient having undergone radiotherapy in combination with a second cancer treatment procedure, in particular chemotherapy and/or surgery.

According to a particular embodiment, the compositions used according to the present invention can be administered to the patient:
before radiotherapeutic treatment combined with chemotherapy, or
before and during radiotherapeutic treatment combined with chemotherapy, or
before and after radiotherapeutic treatment combined with chemotherapy, or
before, during and after radiotherapeutic treatment combined with chemotherapy, or
during radiotherapeutic treatment combined with chemotherapy, or
during and after radiotherapeutic treatment combined with chemotherapy, or
after radiotherapeutic treatment combined with chemotherapy.

According to a particular embodiment, the compositions used according to the present invention can be administered to the patient before radiotherapeutic treatment combined with chemotherapy.

According to a more particular embodiment, the compositions used according to the present invention can be administered to the patient before and during radiotherapeutic treatment combined with chemotherapy.

According to another particular embodiment, the compositions used according to the present invention can be administered to the patient before and after radiotherapeutic treatment combined with chemotherapy.

According to another more particular embodiment, the compositions used according to the present invention can be administered to the patient before, during and after radiotherapeutic treatment combined with chemotherapy.

According to another particular embodiment, the compositions used according to the present invention can be administered to the patient during radiotherapeutic treatment combined with chemotherapy.

According to another more particular embodiment, the compositions used according to the present invention can be administered to the patient during and after radiotherapeutic treatment combined with chemotherapy.

According to another even more particular embodiment, the compositions used according to the present invention can be administered to the patient after radiotherapeutic treatment combined with chemotherapy.

According to a particular embodiment, the compositions used according to the present invention can be administered to the patient:
before radiotherapeutic treatment combined with surgery, or
before and during radiotherapeutic treatment combined with surgery, or
before and after radiotherapeutic treatment combined with surgery, or
before, during and after radiotherapeutic treatment combined with surgery, or
during radiotherapeutic treatment combined with surgery, or
during and after radiotherapeutic treatment combined with surgery, or
after radiotherapeutic treatment combined with surgery.

According to a particular embodiment, the compositions used according to the present invention can be administered to the patient before radiotherapeutic treatment combined with surgery.

According to a more particular embodiment, the compositions used according to the present invention can be administered to the patient before and during radiotherapeutic treatment combined with surgery.

According to another particular embodiment, the compositions used according to the present invention can be administered to the patient before and after radiotherapeutic treatment combined with surgery.

According to an even more particular embodiment, the compositions used according to the present invention can be administered to the patient before, during and after radiotherapeutic treatment combined with surgery.

According to another particular embodiment, the compositions used according to the present invention can be administered to the patient during radiotherapeutic treatment combined with surgery.

According to another more particular embodiment, the compositions used according to the present invention can be administered to the patient during and after radiotherapeutic treatment combined with surgery.

According to another even more particular embodiment, the compositions used according to the present invention can be administered to the patient after radiotherapeutic treatment combined with surgery.

According to a particular embodiment, the compositions used according to the present invention can be administered to the patient:
before radiotherapeutic treatment combined with chemotherapy and surgery, or
before and during radiotherapeutic treatment combined with chemotherapy and surgery, or
before and after radiotherapeutic treatment combined with chemotherapy and surgery, or
before, during and after radiotherapeutic treatment combined with chemotherapy and surgery, or
during radiotherapeutic treatment combined with chemotherapy and surgery, or
during and after radiotherapeutic treatment combined with chemotherapy and surgery, or
after radiotherapeutic treatment combined with chemotherapy and surgery.

According to a particular embodiment, the compositions used according to the present invention can be administered to the patient before radiotherapeutic treatment combined with chemotherapy and surgery.

According to a more particular embodiment, the compositions used according to the present invention can be administered to the patient before and during radiotherapeutic treatment combined with chemotherapy and surgery.

According to another particular embodiment, the compositions used according to the present invention can be administered to the patient before and after radiotherapeutic treatment combined with chemotherapy and surgery.

According to an even more particular embodiment, the compositions used according to the present invention can be administered to the patient before, during and after radiotherapeutic treatment combined with chemotherapy and surgery.

According to another particular embodiment, the compositions used according to the present invention can be administered to the patient during radiotherapeutic treatment combined with chemotherapy and surgery.

According to another more particular embodiment, the compositions used according to the present invention can be administered to the patient during and after radiotherapeutic treatment combined with chemotherapy and surgery.

According to another even more particular embodiment, the compositions used according to the present invention can be administered to the patient after radiotherapeutic treatment combined with chemotherapy and surgery.

The administration schedule of the compositions used according to the present invention can vary and be adapted depending on the extent of the existing or foreseeable mucositis in the cancer patient undergoing anti-cancer treatment or about to undergo anti-cancer treatment. The use of a polyamines-depleted diet can comprise several phases during which the exogenous polyamines supply is:
entirely provided by the compositions used according to the invention,
mostly provided by the compositions used according to the invention,
partially provided by the compositions used according to the invention.

By “entirely”, is meant the fact that the patient's food is restricted to the compositions used according to the invention. No food other than the compositions of the invention are included in the patient's diet. During this phase, the polyamines depletion is at its maximum.

By “mostly”, is meant the possibility of introducing into the patient's diet a breakfast comprising foods with a low-polyamines content. The remainder of the daily food ration is provided by the compositions of the invention.

By “partially”, is meant the possibility of introducing into the patient's diet a breakfast and at least one solid meal comprising foods with a low polyamines content. The remainder of the daily food ration is provided by the compositions of the invention.

According to a particular embodiment, the compositions used according to the present invention are administered to the patient according to the following schedule:

(i) administration of a first dose of said composition during a first period of time and, consecutively,
(ii) administration of a second dose of said composition during a second period of time, the second dose not exceeding the first dose and, consecutively,
(iii) administration of a third dose of said composition during a third period of time, the third dose not exceeding the second dose.

According to another particular embodiment, the compositions used according to the present invention are administered to the patient according to the following schedule:

(i) administration of a first dose of said composition during a first period of time and, consecutively,
(ii) administration of a second dose of said composition during a second period of time, the second dose exceeding the first dose and, consecutively,
(iii) administration of a third dose of said composition during a third period of time, the third dose exceeding the second dose.

According to yet another particular embodiment, the compositions used according to the present invention are administered to the patient according to the following schedule:

(i) administration of a first dose of said composition during a first period of time and, consecutively,
(ii) administration of a second dose of said composition during a second period of time, the second dose being equal to the first dose and, consecutively,
(iii) administration of a third dose of said composition during a third period of time, the third dose being equal to the second dose.

According to yet another particular embodiment, the compositions used according to the present invention are administered to the patient according to the following schedule:

(i) administration of a first dose of said composition during a first period of time and, consecutively,
(ii) administration of a second dose of said composition during a second period of time, the second dose being adjusted depending on the patient's reaction to the first dose and, consecutively,
(iii) administration of a third dose of said composition during a third period of time, the third dose being adjusted depending on the patient's reaction to the second dose.

By “patient's reaction”, is meant their physiological ability to benefit from a low-polyamine diet. Two possible cases can be envisaged: either the grade of the mucositis remains unchanged after administration of a first dose of the composition and in this case, the second dose of the composition is revised upwards, or the grade of the mucositis is reduced after administration of a first dose of the composition and in this case, the second dose of the composition can be revised downwards. The same type of reasoning applies to administration of the third dose. Thus, the practitioner has a great deal of latitude in the administration schedule of the compositions of the invention.

When the three doses are identical, this amounts to administering a single dose to the patient over a period determined by the practitioner.

According to a particular embodiment of the invention, the first period of time varies from 7 to 14 days, in particular 7 days.

According to a particular embodiment of the invention, the second period of time varies from 14 to 21 days, in particular 14 days.

According to a particular embodiment of the invention, the third period of time varies from 28 to 63 days, in particular 63 days.

According to a more particular embodiment of the invention, the first period of time corresponds to the phase preceding the treatment of the patient with radiotherapy.

According to a more particular embodiment of the invention, the second period of time corresponds to the first two weeks of the treatment of the patient with radiotherapy.

According to a more particular embodiment of the invention, the third period of time corresponds to the period extending from the third week to the end of the treatment of the patient with radiotherapy, the abovementioned third period being able to be increased by 4 weeks following discontinuation of said treatment with radiotherapy.

According to an even more particular embodiment of the invention, the compositions used according to the present invention are administered to the patient according to the following schedule:

(i) administration of a first dose of the composition of the invention during the phase preceding the treatment of the patient with radiotherapy and,
(ii) administration of a second dose of the composition of the invention during the first two weeks of treatment of the patient with radiotherapy and,
(iii) administration of a third dose of the composition of the invention over a period of time ranging from the third week to the end of treatment of the patient with radiotherapy, the abovementioned third period being able to be increased by 4 weeks following discontinuation of said treatment with radiotherapy.

According to an even more particular embodiment of the invention, the compositions used according to the present invention are administered to the patient according to the following schedule:

(i) administration of a first dose of the composition of the invention during the 7 to 14 days, in particular 7 days, preceding the treatment of the patient with radiotherapy and,
(ii) administration of a second dose of the composition of the invention during the first 14 to 21 days, in particular the first 14 days, of the treatment of the patient with radiotherapy and,
(iii) administration of a third dose of the composition of the invention over a period of time varying from 28 to 63 days, in particular 63 days up to the end of the treatment of the patient with radiotherapy, the abovementioned third period being able to be increased by 4 weeks following discontinuation of said treatment with radiotherapy.

According to a particular embodiment of the invention, the first dose of polyamines varies from 40 μg to 160 μg of polyamines/d.

According to a particular embodiment of the invention, the second dose of polyamines varies from 30 μg to 120 μg of polyamines/d.

According to a more particular embodiment of the invention, the third dose of polyamines varies from 10 μg to 80 μg of polyamines/d.

According to another particular embodiment of the invention:
the first dose of polyamines varies from 10 μg to 80 μg of polyamines/d,
the second dose of polyamines varies from 30 μg to 120 μg of polyamines/d, and
the third dose of polyamines varies from 40 μg to 160 μg of polyamines/d.

According to yet another particular embodiment of the invention:
the first dose of polyamines varies from 10 μg to 160 μg of polyamines/d,
the second dose of polyamines varies from 10 μg to 160 μg of polyamines/d, and
the third dose of polyamines varies from 10 μg to 160 μg of polyamines/d.

The depletion of polyamines can significantly potentialize the anti-proliferative effects of the conventional anti-tumour drugs (methotrexate, cyclophosphamide, vindesine, etc.) while extending patients' survival time and can make it possible to reduce the quantities of drugs administered while preserving the same anti-tumour effect (Quemener et al., Anticancer Research, 1992, 12.1447-1454).

According to a particular embodiment, the compositions used according to the invention contain, per gram of composition, less than 600 picomoles of biologically active polyamines and optionally at least one anti-tumour agent as a combination preparation for simultaneous or separate use or use spread over time in the prevention or treatment of pathologies of the skin and mucous membranes induced by radiotherapy in a patient.

The anti-tumour agent can be chosen from the following, without being limited to:

abraxane, abarelix, aldesleukin, alemtuzumab, alitretinoin, allopurinol, altretamine, anastrozole, arsenic trioxide, asparaginase, azacitidine, bevacizumab, bexarotene, bicalutamide, bleomycin, bortezomib, intravenous busulphan, oral busulphan, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, 5-fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alpha 2a, irinotecan, lapatinib ditosylate, lenalidomide, letrozole, leucovorin, leuprolide acetate, levamisole, lomustine, mechlorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, sorafenib, streptozocin, sunitinib, sunitinib maleate, tamoxifen, taxol, taxotere, temozolomide, teniposide, testolactone, thalidomide, thioguanine, thiotepa, topotecan, toremifene, tositumomab, trastuzumab, tretinoin, tykerb, uracil mustard, valrubicin, vinblastine, vincristine, vinorelbine, vorinostat, and zoledronate.

### EXPERIMENTAL PART

### Examples of Results Obtained from Clinical Trials on Patients

### Indication

Patients having an ENT (Ear, Nose and Throat) neoplasia who should benefit from exclusive radiotherapy or concomitant radiochemotherapy.

### Patient No. 1:

Infiltrating, keratinizing, well-differentiated epidermoid carcinoma, developed at the expense of the glosso-tonsillar sulcus, classified T3 or even T4 N2c M0 (lesion involving the posterior third of the mobile tongue, the glosso-tonsillar sulcus rising over the whole of the tonsillar fossa, the anterior pillar, the posterior pillar in ulcerative and vegetative form and continuing in serpiginous form opposite the entire soft palate and uvula, clinical N0, bilateral adenopathy on the PET scan).

### Protocol Used:

### Castase™ and Radiochemotherapy:

Castase™, marketed by the company Nutrialys, is a product in the form of drink or cream with a very low polyamines content. Castase™ is an example of a food composition with a low content of biologically “active” polyamines as defined in this invention. Castase™ makes it possible to significantly reduce the consumption of polyamines, up to 8,000 times the highest daily consumption of these tumour growth factor molecules. Castase™ thus promotes better management of patients undergoing anti-cancer treatment, by improving their quality of life and supplementing the action of the chemotherapy.

Classified among Dietary Foods for Special Medical Purposes (DFSMPs) according to European Regulations (Directive 1999/21/EC), Castase™, developed by Nutrialys (Nutrialys Medical Nutrition, Parc EDONIA, Batiment C, rue de la Terre Victoria, 35760 St Grégoire, France), is presented in the form of drink or cream, high-calorie or with a normal level of calories, and high-protein or with a normal level of protein, with a very low polyamines content. They are intended for adults, apart from during pregnancy and breastfeeding.

The characteristics of Castase™: DFSMP with a low polyamine content, rich in Omega 3 fatty acids, gluten-free, lactose-free. The products are available in 200 ml cans or in 200 g pots with different flavours.

It is recommended to administer Castase™ according to a programme. The nutritional programme corresponds to a diet during which a patient's usual food is replaced by a food with a very low polyamines content, Castase™, and then supplemented.

The programme takes place in several phases, and can reach a total duration of 10 to 14 weeks:

1—A first period (“intensive” diet) of 7-14 days during which the patient consumes only Castase™ products. Only breakfast can be made up of tea, coffee, white bread, rusk, butter, cornflakes, puffed rice. This first period makes it possible to reduce the level of polyamines present in the organism.
2—A second period (“standard” diet) of two to three weeks during which the patient consumes both Castase™ products, breakfast (as suggested above) and a meal made up of foods with a reduced polyamines content, based on a Nutrialys™ foods Guide. This second period makes it possible to extend the nutritional programme with a very low polyamines content, by resuming solid food chosen to supplement the Castase™ products (with a low polyamines content).
3—A third period (“maintenance” diet) of 7 to 9 weeks during which the patient consumes both Castase™ products, breakfast (as suggested above) and two meals made up of foods with a reduced polyamines content, based on a Nutrialys™ foods Guide. This third period makes it possible to extend the nutritional programme with a very low polyamines content, by resuming solid food chosen to supplement the Castase™ products (with a low polyamines content).

### Treatment Schedule:

### Pre-Radiotherapy Phase:

First week of “intensive” diet: five high-calorie, high-protein Castase™ products per day: four 200 ml Castase™ drinks+one 200 g Castase™ cream+a breakfast* with a low polyamines content (according to the Nutrialys™ foods Guide). The “intensive” diet provides the patient with at least 52 μg of polyamines/day in the compositions of the invention and 1.2 mg of polyamines/day in the food with a low polyamines content.

| | CASTASE ® drink and cream | | | Standard |
|---|---|---|---|---|
| | “Intensive” | “Standard” | “Maintenance” | food |
| Products | diet | diet | diet | — |
| Number of | 5 | 4 | 3 | — |
| products | | | | |
| advised per day | | | | |
| Calorie intake from | 1500 | 1200 | 900 | — |
| CASTASE ® | | | | |
| products | | | | |
| (kcal/day) | | | | |
| Polyamines | 357 | 286 | 214 | — |
| intake from | | | | |
| CASTASE ® | | | | |
| products | | | | |
| (nmol/day) | | | | |
| Polyamines | 52 | 42 | 31 | — |
| intake from | | | | |
| CASTASE ® | | | | |
| products | | | | |
| (μg/day) | | | | |
| Calorie intake | 500 | 800 | 1100 | 2000 |
| from | | | | |
| foods (kcal/day) | | | | |
| Polyamines | 8 | 29 | 39 | 500 |
| intake from | | | | |
| foods (μmol/day) | | | | |
| Polyamines | 1.2 | 4.2 | 5.7 | 72.6 |
| intake from | | | | |
| foods (mg/day) | | | | |
| Specific nature | With a | With a | With a | Stan- |
| of the | low | low | low | dard |
| food | poly- | poly- | poly- | 250 |
| | amine | amine | amine | nmol/ |
| | content | content | content | kcal |
| | 16 nmol/ | 35.5 nmol/ | 35.5 nmol/ | |
| | kcal | kcal | kcal | |
| Comparison | — | ×3.5 | ×4.8 | ×60.5 |
| with “intensive” | | | | |
| programme | | | | |

### Radiotherapy or Radiochemotherapy Phase:

Second and third weeks of “standard” diet: four Castase™ products per day (three 200 ml Castase™ drinks+one 200 g Castase™ cream)+breakfast*+one meal* with a low polyamines content (according to the Nutrialys™ foods Guide). The “standard” diet provides the patient with at least 42 μg of polyamines/day in the compositions of the invention and 4.2 mg of polyamines/day in the food with a low polyamines content.

“Maintenance” diet up to the end of radiotherapy or radiochemotherapy then continuing for 1 month after the end of radiotherapy or radiochemotherapy: three Castase™ products per day (two 200 ml Castase™ drinks+one 200 g Castase™ cream)+breakfast*+one or two meals* with a low polyamines content (according to the Nutrialys™ foods Guide). The “maintenance” diet provides the patient with at least 31 μg of polyamines/day in the compositions of the invention and 5.7 mg of polyamines/day in the food with a low polyamines content. * to be adjusted depending on the patient's nutritional requirements.

It is considered that a standard diet contains on average approximately 500 μmol per day of polyamines, i.e. a quantity of 73 mg.

Radiochemotherapy Protocol with Patient No. 1:

The patient is treated with concomitant radiochemotherapy by delivering 70 Gy in 35 fractions to the tumour volume over 7 weeks, accompanied by nodal irradiation using 3D conformational radiotherapy following dedicated dosimetry scanning and fusion of the images with the PET scan (Positron emission tomography). The radiotherapy is potentiated by one injection per week of CISPLATIN at a dose of 40 mg/m² over 7 weeks.

### Patient No. 2:

Epidermoid carcinoma of the vocal cords, classified pT1bN0M0 (Scoazec et al., 2010, Annales de pathologic, 30, 2-6).

### Protocol Used:

Castase™ and radiochemotherapy (cf. Patient No. 1).

### Treatment Schedule:

Pre-radiotherapy phase: as for Patient No. 1
Radiotherapy or radiochemotherapy phase: as for Patient No. 1
Radiotherapy protocol with Patient No. 2:

The patient is treated exclusively with radiotherapy of the tumour volume: 70 Gy on the larynx and 50 Gy on the lymph nodes.

### Evaluation of the Therapeutic Effects on the Different Patients:

Nutritional assessment: weight+albuminaemia
Inflammation marker: C-Reactive Protein
Quality of life assessment: score EORTC QLQ-C30 and H&N35
Assessment of mucositis:

Grade 0: Absence.

Grade 1: Pain in the mouth, erythema.

Grade 2: Oral erythema, ulcers, the patient can ingest solid foods.

Grade 3: Mouth ulcers, the patient can ingest only liquid foods.

Grade 4: Feeding by oral route is not possible.

Pain assessment: numerical pain rating scale and analgesic consumption
Assessment of dysphagia, anorexia, asthaenia
Assessment of tolerance: nausea, vomiting, diarrhoea
Standard biological assessment
Assessment of progress of the radiotherapy: doses, postponement of sessions, etc.
Research into dermal toxicity

### Results Obtained:

Observations on the 2 patients cited:

### Patient No. 1:

Good tolerance of the radio- and chemotherapy and of the Castase™ programme throughout the protocol.

Feeding is normal, with no need to transfer to enteral feeding. There is no dysphagia.

The patient develops slight mucositis (grade 2) during and after anti-cancer treatment.

The patient develops very slight dermatitis (grade 1).

### Patient No. 2:

Very good tolerance of the radiotherapy and of the Castase™ programme.

Feeding is normal, with no need to transfer to enteral feeding. The patient's weight is stable.

The patient does not develop mucositis (grade 0-1) during and after anti-cancer treatment.

Monitoring over 10 months shows that there is no recurrence (Nasofibroscopy). (Ganglion=0).

The patient does not develop dermatitis (grade 0) during and after the anti-cancer treatment.

1. Composition comprising a mixture of at least two natural polyamines chosen from spermine, spermidine, putrescine or cadaverine and comprising, per gram of composition, less than 600, in particular less than 400 picomoles of biologically active polyamines, for use in preventing or treating radiotherapy-induced mucositis, salivation problems (dry mouth/pathology of the mucous membranes and salivary glands) and pain on swallowing, that can occur in the case of the highest grades of radiotherapy-induced mucositis, and radiotherapy-induced dermatitis, in a patient, in particular a human or an animal.

2. Composition for use according to claim 1, in which the total quantity of biologically active polyamines ingested per day by the patient does not exceed 0.40 nanomoles per kcal of composition ingested, in particular 0.30 nanomoles per kcal of composition ingested, in particular 0.25 nanomoles per kcal of composition ingested, in particular 0.20 nanomoles per kcal of composition ingested.

3. Composition for use according to claim 1, which contains, per gram of composition, less than 300, in particular less than 200 picomoles of putrescine, less than 50, in particular less than 20 picomoles of spermine, less than 150, in particular less than 100 picomoles of spermidine, less than 100, in particular less than 80 picomoles of cadaverine.

4. Composition for use according to claim 1, in which the radiotherapy is combined either with a chemotherapeutic agent, or with surgery, or with a chemotherapeutic agent and surgery.

5. Composition for use according to claim 1, in which the composition contains, as a percentage of dry weight with respect to the total dry weight: 10% to 35% lipids, 8% to 30% proteins, 35% to 80% carbohydrates, up to 10% of a mixture constituted by vitamins, minerals and electrolytes.

6. Composition for use according to claim 1, in which the composition is enriched with at least one intracellular polyamine synthesis inhibitor, in particular an ornithine decarboxylase, spermidine-spermine N1-acetyltransferase or spermine oxydase inhibitor, or with at least one polyamine transport inhibitor, at a level of at the most 15% by weight with respect to the total dry weight of the composition.

7. Composition for use according to claim 1, in which the composition contains at least one antibiotic and/or is enriched with vitamins.

8. Composition for use according to claim 1, containing carbohydrates, said carbohydrates belonging to the group comprising the glucose polymers, maltodextrins, saccharose, modified starches, glucose monohydrate, dehydrated glucose syrup, glycerol monostearate and mixtures thereof, or containing proteins, said proteins belonging to the group comprising soluble milk proteins, soya proteins, serum peptides, powdered egg white, potassium caseinate, non-phosphorylated peptides, casein peptides, mixed caseinate, soya isolate and mixtures thereof, or containing lipids, said lipids belonging to the group comprising butter oil, groundnut oil, medium chain triglycerides, grape seed oil, soya oil, evening primrose oil and mixtures thereof, or said lipids being constituted by a mixture of at least one oil of animal origin, at least one oil of vegetable origin and glycerol stearate.

9. Composition for use according to claim 1, in which said composition constitutes a human being's daily food ration and in that it comprises:
from 75 g to 500 g of carbohydrates,
from 20 g to 185 g of lipids,
from 20 g to 225 g of proteins,
vitamins, minerals and electrolytes in quantities sufficient to meet the daily nutritional requirements of a human being,
and optionally an intracellular polyamine synthesis inhibitor at a level of less than 50 g and preferentially at a level of 0.3 to 10 g per day.

10. Composition for use according to claim 1, in which said composition is a fraction of a human being's daily food ration and in that it comprises:
from 75/X g to 500/X g of carbohydrates,
from 20/X g to 185/X g of lipids,
from 20/X g to 225/X g of proteins,
vitamins, minerals and electrolytes in quantities sufficient to meet the daily nutritional requirements of a human being,
and optionally an intracellular polyamine synthesis inhibitor at a level of less than 50/X g and preferentially at a level of 0.3/X to 10/X g per day, and X being an integer comprised between 2 and 8 and corresponding to the number of rations that must be ingested by the patient in order to satisfy their daily nutritional requirements.

11. Composition for use according to claim 1, said composition being able to be administered to the patient:
before the radiotherapeutic treatment, or
before and during the radiotherapeutic treatment, or
before and after the radiotherapeutic treatment, or
before, during and after the radiotherapeutic treatment, or
during the radiotherapeutic treatment, or
during and after the radiotherapeutic treatment, or
after the radiotherapeutic treatment.

12. Composition for use according to claim 1, said composition being able to be administered to the patient having undergone radiotherapy in combination with a second cancer treatment procedure, in particular chemotherapy and/or surgery.

13. Composition for use according to claim 12, said composition being able to be administered to the patient:
before the radiotherapeutic treatment combined with chemotherapy, or
before and during the radiotherapeutic treatment combined with chemotherapy, or
before and after the radiotherapeutic treatment combined with chemotherapy, or
before, during and after the radiotherapeutic treatment combined with chemotherapy, or
during the radiotherapeutic treatment combined with chemotherapy, or
during and after the radiotherapeutic treatment combined with chemotherapy, or
after the radiotherapeutic treatment combined with chemotherapy.

14. Composition for use according to claim 12, said composition being able to be administered to the patient:
before the radiotherapeutic treatment combined with surgery, or
before and during the radiotherapeutic treatment combined with surgery, or
before and after the radiotherapeutic treatment combined with surgery, or
before, during and after the radiotherapeutic treatment combined with surgery, or
during the radiotherapeutic treatment combined with surgery, or
during and after the radiotherapeutic treatment combined with surgery, or
after the radiotherapeutic treatment combined with surgery.

15. Composition for use according to claim 12, said composition being able to be administered to the patient:
before the radiotherapeutic treatment combined with chemotherapy and surgery, or
before and during the radiotherapeutic treatment combined with chemotherapy and surgery, or
before and after the radiotherapeutic treatment combined with chemotherapy and surgery, or
before, during and after the radiotherapeutic treatment combined with chemotherapy and surgery, or
during the radiotherapeutic treatment combined with chemotherapy and surgery, or
during and after the radiotherapeutic treatment combined with chemotherapy and surgery, or
after the radiotherapeutic treatment combined with chemotherapy and surgery.

16. Composition for use according to claim 1, in which the composition is administered to the patient according to the following schedule:
(i) administration of a first dose of said composition during a first period of time and, consecutively,
(ii) administration of a second dose of said composition during a second period of time, the second dose being adjusted depending on the patient's reaction to the first dose and, consecutively,
(iii) administration of a third dose of said composition during a third period of time, the third dose being adjusted depending on the patient's reaction to the second dose.
